(19) United States
(12) Patent Application Publication
(10) Pub. No.: US 2016/0287510 A1
(43) Pub. Date: Oct. 6, 2016

(54) Bust d'Odor Deodorant

(71) Applicant: ISMAIL MENSAH DWUMFOUR-POKU, MESQUITE, TX (US)

(72) Inventor: ISMAIL MENSAH DWUMFOUR-POKU, MESQUITE, TX (US)

(21) Appl. No.: 14/674,176

(22) Filed: Mar. 31, 2015

(51) Int. Cl. A61K 8/97; A61Q 15/00

(52) U.S. Cl. CPC A61K 8/97; A61Q 15/00

(57) ABSTRACT

Bust d'Odor Deodorant is a clear liquid roll-on deodorant for men and women with lemon juice and botanical extracts (honeysuckle and orange lemongrass). It is designed to remove strong underarm odor that may last up to 48 hours. There are no antiperspirants, which is the whole objective of this invention. It does not prevent sweating as do antiperspirants, because sweating is good for the human body. To prevent underarm sweat is to keep toxins or waste products in the body; and adding the chemicals in antiperspirants on top of this makes it very dangerous and unhealthy for the human body. This is a breakthrough invention for mankind, health-wise. It has no side effects except for minor itch caused by cuts from shaving. It leaves no residue or gummy, sticky feeling under armpit, and does not stain or discolor clothing.

### BACKGROUND OF INVENTION

The medicinal value of lemon and its hidden properties as an odor remover, metal polisher, and many other uses including hydration have been established for centuries in Africa, Asia, and other parts of the world. Having used this natural product as an underarm cleanser for many, many, years growing up with parents and grandparents, and now with my immediate family, has been a wholesome and healthful experience. Many of the cancers associated with the use of deodorants with antiperspirants could have been prevented if the healing powers and properties of lemon juice as an odor remover were put to use commercially. With this new invention, one of the greatest benefits to society is to improve the quality of life, by not only prolonging life, but also to mitigate the dangers associated with the use of antiperspirants/parabens. As an Epidemiologist and a Ph.D student in Epidemiology, my vision and mission is to mitigate, if not completely eradicate, breast cancer prevalence and other chronic and infectious diseases afflicting humanity today. It will be immoral not to share this precious gift my grandparents gave me, knowing that the health benefits are immense and significant for all.

### BRIEF SUMMARY OF THE INVENTION

Antiperspirants/parabens are human carcinogens known to cause breast cancer and other diseases, according to evidence-based research. Bust d'Odor Deodorant is an excellent healthy substitute for underarm cleansing designed to help people live a long, productive life without the hazards of any of these toxic chemicals or impuritants. While it does not prevent one from sweating, which is an essential body mechanism designed to help our bodies stay cool, it does remove strong underarm odor for up to 48 hours. The skin is the largest organ of our body and some of its important roles are to expel toxins through sweating, which supports proper immune function and helps prevent diseases related to toxic overload. Why hold these toxins in our body by using antiperspirants to keep us from sweating, which add more dangerous toxic chemicals, further compounding the problem? Besides maintaining proper body temperature and keep you from overheating, sweating kills viruses and bacteria that cannot survive in temperatures above 98.6 degrees Fahrenheit. Sweating also helps clean the pores which helps eliminate pimples and acne. The answer to these obnoxious chemicals and the beauty of sweating, the body's excellent mechanism of excretion and thermoregulation, is the result of Bust d'Odor Deodorant.

### DETAILED DESCRIPTION OF THE INVENTION

Bust d'Odor Deodorant is clear, unscented roll-on deodorant for men and women that eliminates strong underarm odor. It has no antiperspirants, the dreaded toxic chemicals in deodorants such as aluminum-zirconium, aluminum chloride, and aluminum chlorohydrate compounds found to be very unhealthy. It contains 100% pure, fresh lemon juice and botanical extracts such as lemongrass blend and honeysuckle blend to prolong shelve life. It is for external use only, applied to underarm to remove underarm odor. It has proven to be very effective with no side effects and can last up to 48 hours, even after profuse sweating and shower without further application. One of the biggest advantages is that it is clear and does not leave stains in garments or gummy and sticky residue in the armpit or clothes, and does not smell, saving you trips and money from the cleaners.

### TECHNICAL FIELD

Ingredients: 100% Lemon juice (no water)

Preservatives: Orange-Lemongrass Blend; Honeysuckle Blend

2.0 Fl. Oz (59 mL) of lemon juice (100% pure; no concentrate), plus 1 cc drop each of lemongrass and honeysuckle blends

What is being claimed are:

1. The originality of the conceptual idea of the use of lemon juice and botanical extracts for preservatives as a healthy alternative for deodorants.

2. The ingredients (lemon juice, lemongrass blend, honeysuckle blend) and the method of mixing these ingredients; % concentration of preservatives per mL of lemon juice.

3. Lemon Juice for use as deodorant to cure strong underarm odor
